(19) United States
(12) Patent Application Publication
(10) Pub. No.: US 2026/0102553 A1
(43) Pub. Date: Apr. 16, 2026

(54) Cleansing Device and Method

(71) Applicant: Bradley H. Weiss, Los Angeles, CA (US)

(72) Inventor: Bradley H. Weiss, Los Angeles, CA (US)

(21) Appl. No.: 19/354,544

(22) Filed: Oct. 9, 2025

Related U.S. Application Data
(60) Provisional application No. 63/706,900, filed on Oct. 14, 2024.

(51) Int. Cl. A61M 3/02

(52) U.S. Cl. CPC A61M 3/0262; A61M 3/0279

(57) ABSTRACT

A cleansing system may comprise a bulb, wherein the bulb is configured to be collapsible; a nozzle having a first end that removably interfaces with the bulb; and a back-flow prevention cap that interfaces with the first end of the nozzle.

### CROSS-REFERENCE TO RELATED APPLICATIONS

This application claims priority to and the benefit of U.S. Provisional Ser. No. 63/706,900 filed on Oct. 14, 2024, and entitled “Cleansing Device and Method,”which is incorporated in its entirety for all purposes.

### FIELD

This disclosure generally relates to a cleansing device, and more particularly, to a collapsible cleansing device.

### BACKGROUND

The more common systems meant for douching may include, for example, a bulb with a nozzle or a hose that connects to a water source (e.g., shower head). Such systems may be particularly useful for anal douching. The bulb and nozzle solution has many disadvantages. For example, such systems are often of a bulky size and shape. Moreover, existing systems often include a bulb that retains its shape and size when not in use. Such systems are often inconvenient to carry in a day bag because the systems take up a lot of storage space. Moreover, such systems are not easy to store and keep discreet, which is particularly important for these types of douching systems that people want to keep private. It is very difficult to mask the identity of such systems, which can lead to awkward moments if discovered by other parties (e.g., airport TSA agents) or guests/family at your home.

Furthermore, such systems are typically difficult to clean. For example, many current solutions have small connection points where the bulb and nozzle meet, which makes it really difficult to wash the inside. Additionally, existing systems have a problem with the back-flow of water. In particular, many existing systems do not have mechanisms to prevent the flow of liquid back into the bulb during use. Such a back-flow problem may increase the possibility of bacteria entering the system.

### SUMMARY

In various embodiments, a cleansing system may comprise a bulb, wherein the bulb is configured to be collapsible; a nozzle having a first end that removably interfaces with the bulb; and a back-flow prevention cap that interfaces with the first end of the nozzle.

The back-flow prevention cap may removably interface with the first end of the nozzle. The back-flow prevention cap may include an opening, wherein the opening temporarily opens in response to an increase of fluid pressure, and wherein the opening temporarily closes to restrict fluid flow in response to a decrease of the fluid pressure. The system may also include a ring set within an opening of the bulb, wherein the ring includes internal threads that engage with external threads of the nozzle. The first end of the nozzle may removably interface with the bulb via a threaded interface. The bulb may include a grab device. The system may further comprise at least one of a cleaning kit, an antibacterial solution or a scrubbing wand.

### BRIEF DESCRIPTION OF THE DRAWINGS

The accompanying drawings, wherein like numerals depict like elements, illustrate exemplary embodiments of the present disclosure, and together with the description, serve to explain the principles of the disclosure. In the drawings:

FIG. 1 shows an exemplary cross-section of an expanded bulb, in accordance with various embodiments.

FIG. 2 shows an exemplary nozzle, in accordance with various embodiments.

FIG. 3 shows an exemplary nozzle with back-flow prevention cap, in accordance with various embodiments.

FIG. 4 shows an exemplary back-flow prevention cap with slit, in accordance with various embodiments.

FIG. 5 shows an exemplary ring, in accordance with various embodiments.

FIG. 6 shows an exemplary constructed system with the expanded bulb and nozzle, in accordance with various embodiments.

FIG. 7 shows an exemplary collapsed bulb with the nozzle removed, in accordance with various embodiments.

FIG. 8 shows an exemplary cross-section of the collapsed bulb with the nozzle removed, in accordance with various embodiments.

### DETAILED DESCRIPTION

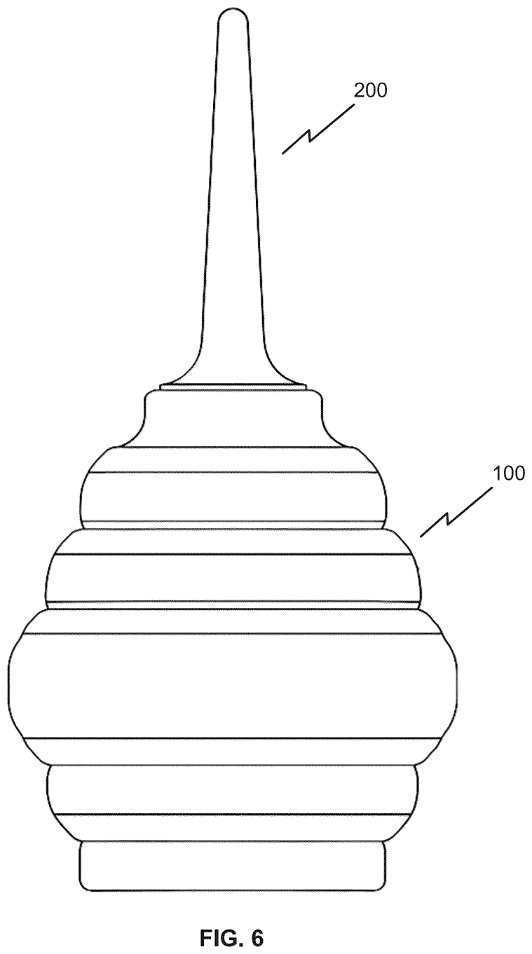

The cleansing system may include various components such as, for example, a bulb 100, ring 500, nozzle 200, and back-flow prevention cap 400. The components of the system may be comprised of any material, be of any shape and any dimensions. An example of the constructed system is shown in FIG. 6, having the nozzle 200 inserted into the bulb 100. The total assembled height of the system may be about 6.627 inches.

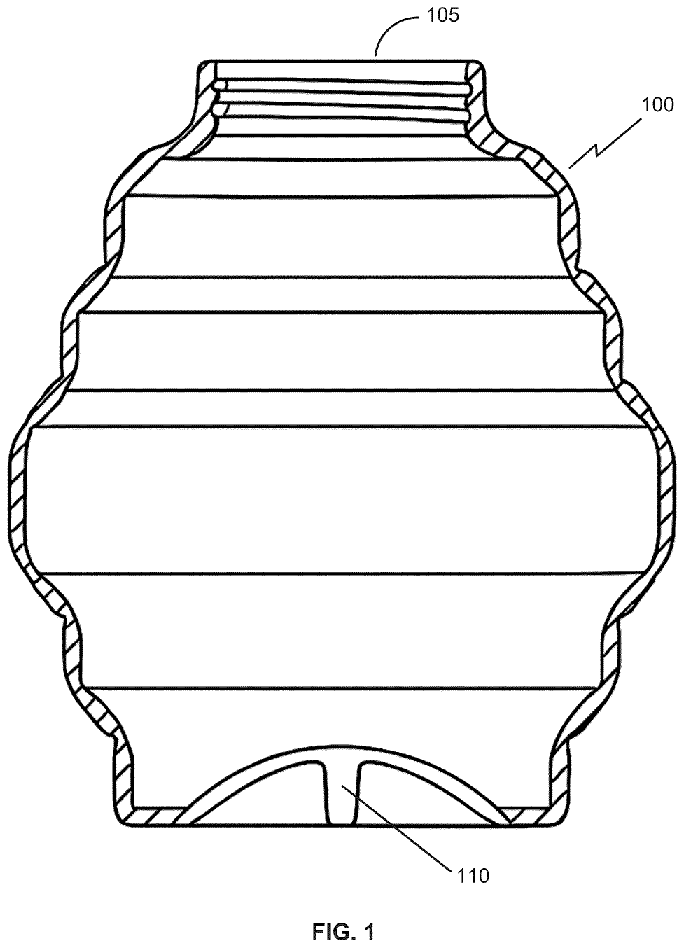

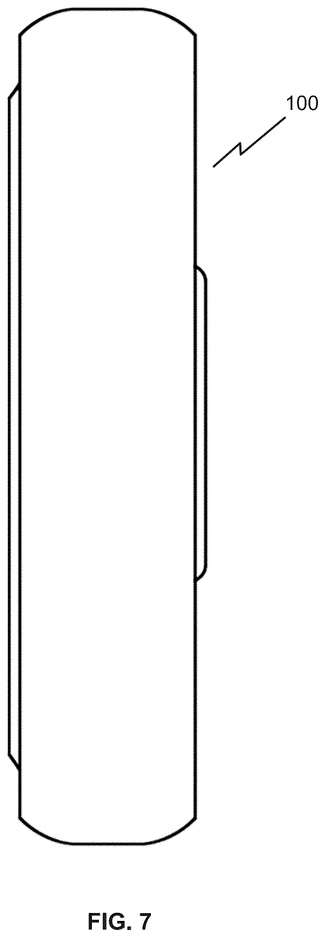

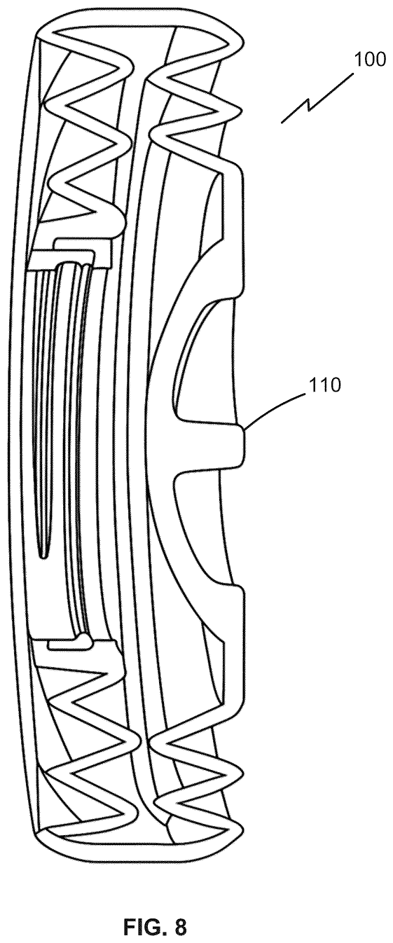

In various embodiments, as shown in FIG. 1, the bulb 100 may be comprised of any material such as, for example, silicone. The bulb 100 may be about 3.25 inches in diameter at the widest point; about 0.777 inches collapsed height; and about 3.702 inches expanded height. Measurements may vary due to the nature of the material. The bulb 100 may be collapsible, as shown in FIGS. 7 and 8. As shown in FIG. 6, the bulb 100, when expanded, holds its general shape on its own and may hold up to approximately 264 ml of water. The bulb 100 may be very malleable and may be configured to be squeezed by hand to push water out through the nozzle 200. When the bulb 100 is separated and empty, the top and bottom fold into itself like an accordion or into a “bellows” shape. The opening 105 in the bulb 100 connection point is about 1.070 inches in diameter, which makes it easy to get a finger inside to clean by hand.

The opening 105 in the bulb 100 may include a threaded surface. The threaded surface may engage with the threaded surface of one end of the nozzle 200. The bulb 100 may include an inside threaded surface and the nozzle 200 may include an outside threaded surface, such that the bulb 100 receives the nozzle 200. However, the threaded surfaces may be opposite such that the bulb 100 may include an outside threaded surface and the nozzle 200 may include an inside threaded surface, such that the nozzle 200 receives the bulb 100. The bulb 100 may include a ring 500 with threads that mate with the removable nozzle 200. The threaded interfaces help to create a water-tight seal.

In various embodiments, the bottom of the bulb 100 may include a grab device 110 to allow the user's fingers to hold and manipulate the bulb 100. The grab device 110 may be any inlet, insert and/or projection. The bulb 100 may also include surfaces that include logos and/or text.

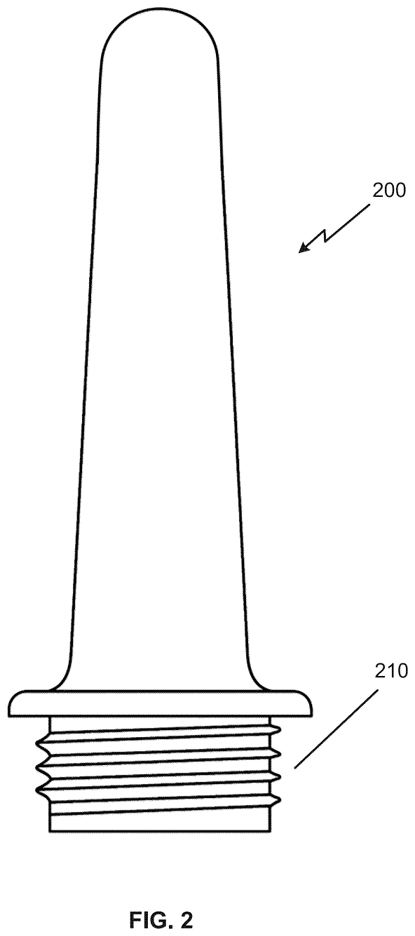

In various embodiments, as shown in FIG. 2, the nozzle 200 may be about 3.25 inches in length and the base of the nozzle 200 may be about 0.866 inches in width. The total width of the nozzle 200 is about 1.220 inches and the width of the opening is about 1.070 inches. The nozzle 200 may be comprised of transparent acrylic (a hard plastic), polycarbonate and/or lead free. In various embodiments, the nozzle 200 may include a threaded base 210 that screws into the bulb 100 to create an air-tight seal and/or water-tight seal.

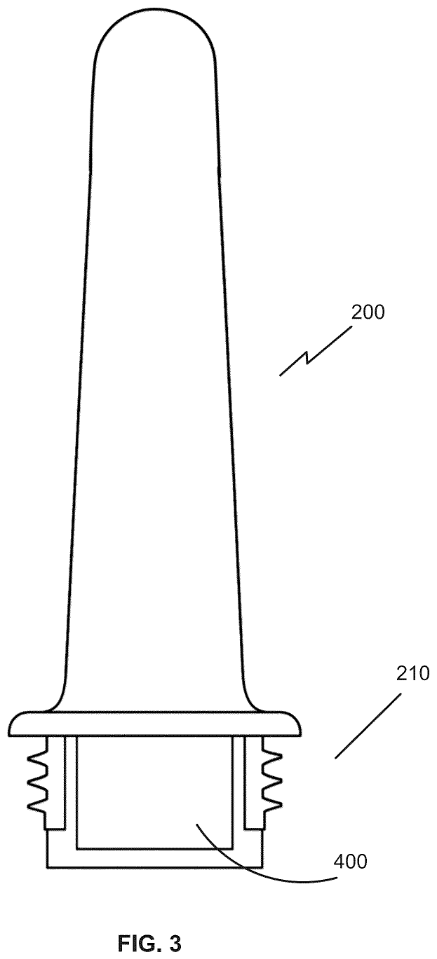

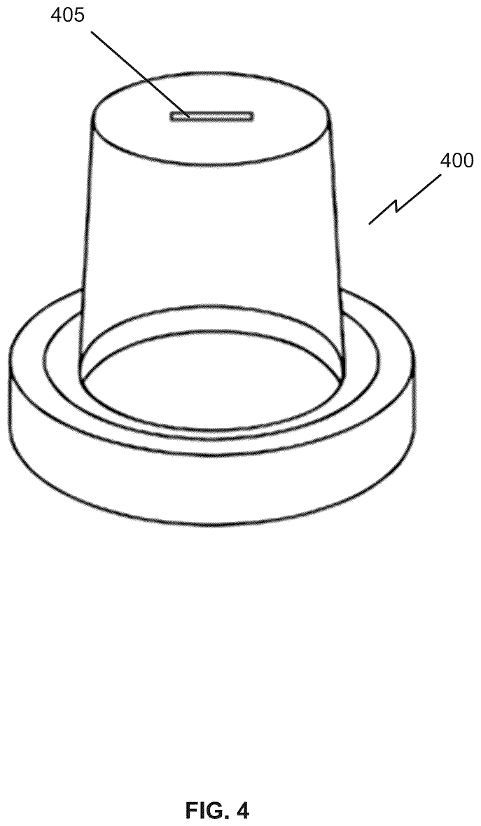

As shown in FIG. 3, the back-flow prevention cap 400 may be removably attached (or removably received into) the bottom of the nozzle 200 below the threaded portion. The interface between the back-flow prevention cap 400 and the bottom of the nozzle 200 creates a water-tight seal. In various embodiments, as shown in FIGS. 3 and 4, the back-flow prevention cap 400 may be any type of membrane insert. The back-flow prevention cap 400 may be removably attached (or removably received into) the bottom of the nozzle 200 below the threaded portion. The back-flow prevention cap 400 may be about 0.872″ diameter and about 0.661″ height. The back-flow prevention cap 400 material may be 100% silicone, or mixed with any other material(s). The back-flow prevention cap 400 may include a slit 405 (or other type of opening) anywhere on the cap 400. Upon applying increased fluid pressure, the slit may open to allow fluid to flow out of the bulb 100 and into the nozzle 200. However, the slit 405 may close when the pressure is released. In other words, back-flow prevention cap 400 may help prevent the flow of air, water or other fluids back into the unit, upon a reduction of pressure against the bulb 100 during use. In various embodiments, the back-flow prevention cap 400 may include a small cylindrical piece of silicone with a slit 405 in the middle of one side that snaps onto a groove in the base of the nozzle 200. The back-flow prevention cap 400 is optional, as the system will function with or without the back-flow prevention cap 400 in place. In various embodiments, the system may include a round disk that sits in the nozzle 200. In various embodiments, the system may include a cylindrical piece that snaps on due to the orientation of the nozzle 200 and bulb 100 connection.

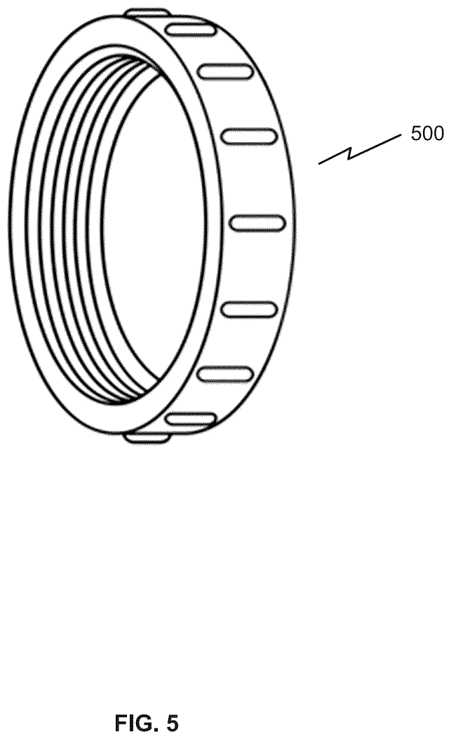

In various embodiments, as shown in FIG. 5, ring 500 may be inserted inside opening 105 in the bulb 100 to provide an interface with nozzle 200. Ring 500 may be bonded to the silicone surface using adhesive. Because bulb 100 may be comprised of malleable silicone and the nozzle may be comprised of a hard material, a hard surface may help with improving the interface. For example, ring 500 is sandwiched and glued into the top of the inside surface of the bulb 100, so that the nozzle 200 grips to a hard surface, as nozzle 200 is being screwed into bulb 100.

The benefits of the system include providing for a more enjoyable experience during the cleansing process and to cater to the needs of users when not in use. By allowing the system to collapse and tuck away into a small pouch, users can discreetly keep the system with them at all times, or store it without people accidentally discovering it. By making the bulb 100 opening 105 almost an inch wide, not only is it easy to fill with water, but a user can comfortably use their finger to scrub the inside with soap and water making for a more hygienic solution.

Furthermore, with the inclusion of the optional back-flow prevention disk 400, users have less worry about potentially tainted liquid re-entering the bulb 100.

In various embodiments, when the two main components are separated, the components can be stored together side-by-side in a discreet travel pouch. The method of use includes the nozzle 200 being configured to be inserted into the user's body. The bulb 100 is configured to hold water (or other liquid) during use and push the water through the nozzle 200 to enter the user's body. Once the user squeezes the bulb 100, the user can then remove the unit from their body, refill, and repeat as needed. The two main components rely on each other for proper use. As mentioned, the base of the nozzle 200 is threaded and screws into the top of the bulb 100 creating a seal.

The intended use is the user expands the bulb 100 like an accordion by gently pulling the top and bottom in opposite directions. If the user wishes to use the back-flow prevention cap 400, the user can insert it into the nozzle 200 by pushing it in until the cap 400 is secure. The user fills the bulb 100 with water and screws on the nozzle 200. The user may remove remaining air that may be in the system by either giving the bulb 100 a gentle squeeze or by running additional water along the hole in the nozzle 200. The user inserts the system into their anal orifice (or any other orifice) and squeezes the bulb 100 to push the water out of the bulb 100 and into their body. After the user is satisfied with the amount of water inserted, the system is removed and the user may ideally discard the water in a toilet. As the user unscrews the nozzle 200 from the bulb 100, it will restore its shape, and the user can repeat previous steps, if desired. When the user is finished using the system, the user can separate all parts, clean under a faucet or in a dishwasher, and then collapse the bulb 100 to store with the nozzle 200 in the pouch.

In various embodiments, the system may include additional nozzle 200 options to mix and match with the bulb 100. In various embodiments, the nozzle 200 may be of different sizes (e.g., thicker, narrower, longer, tapered). In various embodiments, the components may include different materials. For example, a more bendable silicone option. In various embodiments, the components may be comprised of different colors. In various embodiments, the system may include different bulb 100 sizes with similar functionality and collapsibility. In various embodiments, the system may include a cleaning kit comprising a travel size antibacterial solution and a silicone scrubbing wand. The system may also address the other type of douche system option (hose and attachment), by leveraging expandable hose innovation/design. For example, the system may be used with any hose type devices, collapsible hoses, shower heads and/or other running water sources.

The detailed description of various embodiments herein makes reference to the accompanying drawings, which show various embodiments by way of illustration. While these various embodiments are described in sufficient detail to enable those skilled in the art to practice the disclosure, it should be understood that other embodiments may be realized and that changes may be made without departing from the scope of the disclosure. Thus, the detailed description herein is presented for purposes of illustration only and not of limitation.

Furthermore, any reference to singular includes plural embodiments, and any reference to more than one component or step may include a singular embodiment or step. Also, any reference to attached, fixed, connected, or the like may include permanent, removable, temporary, partial, full or any other possible attachment option. Additionally, any reference to without contact (or similar phrases) may also include reduced contact or minimal contact. It should also be understood that unless specifically stated otherwise, references to “a,” “an” or “the” may include one or more than one and that reference to an item in the singular may also include the item in the plural. Further, all ranges may include upper and lower values and all ranges and ratio limits disclosed herein may be combined.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more. ” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

Systems, methods and apparatus are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S. C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Finally, it should be understood that any of the above described concepts can be used alone or in combination with any or all of the other above described concepts. Although various embodiments have been disclosed and described, one of ordinary skill in this art would recognize that certain modifications would come within the scope of this disclosure. Accordingly, the description is not intended to be exhaustive or to limit the principles described or illustrated herein to any precise form. Many modifications and variations are possible in light of the above teaching.

I claim:

1. A cleansing system comprising:
a bulb, wherein the bulb is configured to be collapsible;
a nozzle having a first end that removably interfaces with the bulb; and
a back-flow prevention cap that interfaces with the first end of the nozzle.

2. The cleansing system of claim 1, wherein the back-flow prevention cap removably interfaces with the first end of the nozzle.

3. The cleansing system of claim 1, wherein the back-flow prevention cap includes an opening, wherein the opening temporarily opens in response to an increase of fluid pressure, and wherein the opening temporarily closes to restrict fluid flow in response to a decrease of the fluid pressure.

4. The cleansing system of claim 1, further comprising a ring set within an opening of the bulb, wherein the ring includes internal threads that engage with external threads of the nozzle.

5. The cleansing system of claim 1, wherein the first end of the nozzle removably interfaces with the bulb via a threaded interface.

6. The cleansing system of claim 1, wherein the bulb includes a grab device.

7. The cleaning system of claim 1, further comprising at least one of a cleaning kit, an antibacterial solution or a scrubbing wand.
